(19) United States
(12) Patent Application Publication
(10) Pub. No.: US 2018/0000995 A1
(43) Pub. Date: Jan. 4, 2018

(54) DEVICE FOR FLUID TRANSFER MADE IN NANOMATERIAL

(71) Applicant: INSTITUCION UNIVERSITARIA SALAZAR Y HERRERA, Medellín (CO)

(72) Inventor: Alejandro Vargas Gutierrez, Medellin (CO)

(21) Appl. No.: 15/543,241

(22) PCT Filed: Jan. 13, 2015
(86) PCT No.: PCT/IB2015/050258

§ 371 Date: Jul. 13, 2017

(51) Int. Cl. A61L 31/04; A61L 29/04; A61M 25/00; A61M 5/00; A61M 25/01

(52) U.S. Cl. CPC A61L 31/049; A61M 25/0043; A61M 5/00; A61L 29/042; A61M 25/0127; A61M 2025/0058

(57) ABSTRACT

A continuously hollow device for the transfer of fluids to the human body is provided, which is made in a nanomaterial derived from latex, which chemical composition in based primarily on fats, waxes and several gummy resins obtained from cytoplasm of lactic cells. This biochemically modified material is capable of adjusting its outer and inner diameter, i.e. it enlarges or shrinks according to the needs of the patient. The device corresponds to a needle or the like (catheter), which allows to have a very small diameter so as to be inserted in the patient (minimally invasive procedure) and after a physical excitation of such nanomaterial, it can be extended once inserted in the body so as to allow the intake or discharge of fluids to or from the body through a catheter, probe or the like.

### CROSS-REFERENCE TO RELATED APPLICATION

This application claims priority to PCT Application No. PCT/IB2015/050258 having a filing date of Jan. 13, 2015, the entire contents of which is hereby incorporated by reference.

### FIELD OF TECHNOLOGY

The following relates to a continuously hollow device for the transfer of fluids to the human body which is made in a nanomaterial derived from latex, which chemical composition in based primarily on fats, waxes and several gummy resins obtained from cytoplasm of lactic cells. This biochemically modified material is capable of adjusting its outer and inner diameter, i.e. it enlarges or shrinks according to the needs of the patient, wherein such device corresponds to a needle or the like, which allows to have a very small diameter so as to be inserted in the patient and then by an excitation of such nanomaterial, it can be extended once inserted so as to allow the intake of fluids to the body by means of a catheter or the like. Thus, the fluid transfer device after being used it can go back to its reduced diameter state so that it can be removed from the patient avoiding thereby the great pain and risk of traumas in the skin, arteries, veins and other organs of the anatomy.

### BACKGROUND

One of the major problems present in Public Health in several countries, is the trauma by accident and/or violence, which makes it necessary in the health centers a large amount of intravenous needles and catheters, in order to perform the suitable treatments to patients in shock state who need to receive fluids intravenously in a fast and effective manner, in order to increase the blood pressure and to stabilize the vital signs.

Similarly, the blood donors and the blood banks have the above mentioned need, at the same time that there is a problem with patients and donors related to pain and the possible complications that can arise in these donation procedures, which translates in a representative limitation when obtaining supplies. Likewise, the health system needs to reduce the medical complications of the patients with trauma so as to reduce the costs in important events in public health.

In this regard, the nanotechnology has allowed in the last years the development of new materials with several physical and chemical properties being usually at large scale, but which are also available now in nanometer-scale devices. In addition, several of the common properties of these materials are sensitive to change and physical variables which are not evident at large scale, which makes these properties to be available to be exploited in new applications in engineering, biotechnology, electronic, health care and other areas. This new area of biomedical knowledge, nanotechnology, aims to the development of minimally invasive devices in order to avoid pain and complications in patients and at the same time, avoids excessive expenses to the health entities for the adverse events of the traditional devices.

Thus, there is a plurality of disclosures in the state of the art related to needle systems than can expand and contract according to the needs, which are useful when performing a procedure on a patient or when introducing some type of fluid therein, such as a drug. Within these disclosures is the document CA 2788391, which refers to a catheter small enough in diameter to be placed minimally invasively into the body of a patient but also can be expanded post-placement to provide a larger diameter placed catheter that supports fluid flow at a rate higher than is possible through the pre-placement reduced-diameter catheter, wherein said catheter is constructed using one or more shape memory polymers and can include one or more sections configured to enhance the flexibility of the section as compared to the other parts of the catheter and thus allow the section to accommodate tight bends and turns when inserted into the body of the patient.

However, embodiments of the invention described in this anteriority features the disadvantage of being a memory polymer material, not based on nanotechnology, which creates a device similar to those already existing but which depends directly on the material response to maintain its original shape, i.e. if the material is worn or it loses its elasticity or ability to go back to its original state, it is locked in a very small diameter which makes it difficult to transfer the fluid at the desired rate.

On the other hand, document US 2012046608 discloses a medical balloon and methods of modifying said balloon by forming a void pattern in its exterior surface and filling the voids with a material, such as a fiber or a nanomaterial, e.g., nanotubes, such as carbon nanotubes, and a matrix material, e.g., a polymer, wherein such medical balloon corresponds specifically to a catheter having an elongated shaft and an inflatable balloon located adjacent to the distal end of the shaft, wherein the catheter contains a coating comprising the nanomaterial, wherein such coating is arranged inside some slots formed in an outer surface of the catheter.

Now, document MX 2012001390 mentions a device of expandable intravenous access, such as an expandable catheter, which facilitates a less intrusive access to medical equipment in the patient, wherein the catheter comprises a ring of fixed diameter for defining the maximum size of the introduced needle, from which a plurality of flexible rods are protruded, these rods being able to displace radially from an initial position to the maximum size of the ring, wherein the catheter consists of a rigid structure composed by metal or polymer elements and a flexible element made of an elastic material.

However, embodiments of the invention defined in this anteriority shows the disadvantage that the device has a rigid structure and the expansion thereof is made through an elastic flexible material, which is undesirable, since two different types of material are required for its manufacture, increasing thereby the production costs.

Finally, document AU 2012308610 is related to an intravascular catheter including a catheter tube having an expandable portion with a plurality of struts wherein each defines an outer surface, wherein the expandable portion is operable between a closed position wherein the expandable portion has a first diameter, and an opened position, wherein the expandable portion has a second diameter that is larger than the first diameter. Similarly, an incising element is provided on the outer surface of at least one of the struts, wherein the incising element has a blade that extends outwardly in a radial direction from the outer surface for creating an incision.

### SUMMARY

According to the above, it is clear that there is a need in the state of the art to design and implement a needle and/or catheter system featuring the characteristics of being manufactured in a nanomaterial derived from latex, but which is biochemically modified in the laboratory such that its original molecular composition changes depending on the response to an external physical stimulus (optical stimulus with LED-type sensor, with a magnetic field, with an electric current or temperature change). This physical stimulus generates a sustained but reversible distancing between the molecules of the material whereby the manufactured device allows to obtain a needle or catheter with a very small diameter at the beginning of the procedure (blood donation, vein or artery canalization, placement of thorax, vesicle, nasogastric catheter, or orotracheal tube in general anesthesia, etc.) so as to cause no pain when introduced in the patient, but that after verifying by the health personnel that the device properly canalized the vein, artery or reached the target organ (lung, bladder, stomach, trachea, etc.), the physical stimulus is activated to increase such diameter once inside the body of the patient, in order to be able to provide liquids, drugs, hemoderivatives, or to draw blood or homoderivatives from donors, to insert a catheter or probe, etc. and reduce thereby the pain feeling and the risk of trauma or infections in the puncture location. Once the object is achieved with the needle or catheter, the health personnel deactivates the physical stimulus (LED light, electrical current, magnetic field, temperature gradient) and therewith, the molecules of the nanomaterial recover its original distance between them, thus achieving the removal of the device with less pain and trauma for the patient.

In this regard, it is clear that the need is based on applying a mixture of new nanotechnology, nano-sensors and developments in plastic materials used in the health industry so as to allow the design of a single size hypodermal needle or probes or catheter that changes its diameter as a response to a specific command, i.e. with the excitation, whether it is optic, magnetic, electric, etc.

### BRIEF DESCRIPTION

Some of the embodiments will be described in detail, with reference to the following figures, wherein like designations denote like members, wherein:

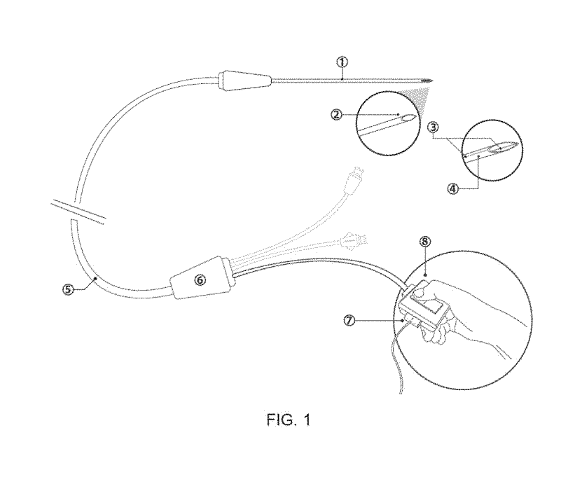

FIG. 1 corresponds to the needle with nanomaterial in small caliber being introduced in the veins or arteries of the patients, wherein the activation of the nanosensor located inside thereof (optical effect by LED light), activates the nanomaterial so its original state (latex molecules bound together to each other with the minimum distance therebetween) is modified by a transitory state consisting of a measurable increase of the distance between the latex molecules which allows the diameter to be increased.

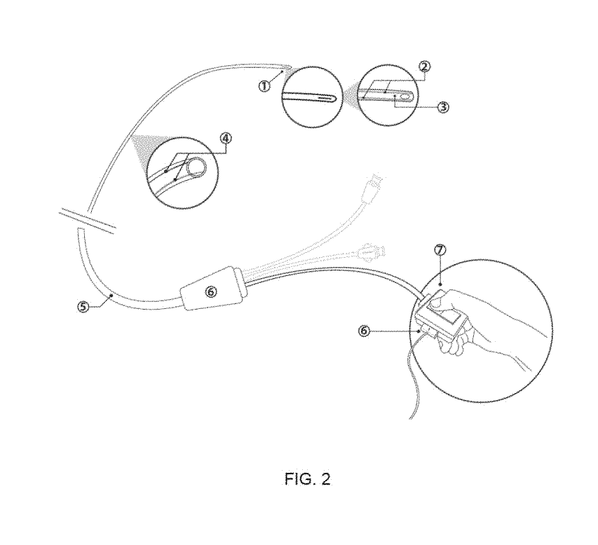

FIG. 2 corresponds to the probe manufactured with nanomaterial in small caliber being introduced in the urethra of patients (vesicle probe), wherein the activation of the nanosensor located inside thereof (optical effect), activates the nanomaterial to increase the diameter of the probe. Note the option of external manipulation of the nanosensor which allows the control of the diameter by the health personnel.

### DETAILED DESCRIPTION

Embodiments of invention corresponds to a device (1) for a variable diameter hypodermal needle (2), wherein the diameter of said needle (2) varies through the use of nanotechnology, since it is manufactured in a latex-derived nanomaterial which chemical composition is fundamentally based in fats, waxes and diverse gummy resins obtained from cytoplasm of lactic cells. This biochemically modified material goes from an original state to a transitory state consisting of a measurable increase of the distance between the latex molecules which allows the diameter to be increased, i.e. it enlarges or shrinks according to the needs of the patient, wherein such device corresponds to a needle or the like, which allows to have a very small diameter to be able to be inserted in the patient and then through an excitation of said nanomaterial, it can be enlarged once inserted so as to allow the intake of fluids to the body through a catheter or the like. Thus, the fluid transfer device after being used can return to its reduced diameter so that it can be removed from the patient avoiding pain and trauma risks in the skin, arteries, veins and other organs of the anatomy.

The fluid transfer device (1) further comprises the following components or parts:
A sensor (3);
A container (5) for storing the liquid to be injected to removed;
A control system (8) directly connected to the sensor (3); and
A power source (7) which provides the necessary power to all the elements.

In an embodiment of the present invention, the expansion of the needle (2) is achieved through a source of light (3), such as an LED source, which is located inside the needle (3) and which can be even thinner than a human hair. Thus, once the light is activated inside the needle, said stimulus allows the molecular excitation of the latex atoms which change its biochemical composition by a distancing therebetween which is macroscopically translated in the diameter enlargement of said needle (2), at the same time, the deactivation of the optical stimulus can contract the diameter, by a change in the organization of the latex nanomaterial molecules inside the needle (2), as well as any catheter or probe (material resilience which allowed its return to the original dimensions).

In an alternative embodiment, the diameter change of the needle (2) is achieved through the temperature change between the environment outside and inside the cavities of the human body where there is a temperature gradient. Thus, when inside the human body, the temperature is greater than the room temperature, whereby the inside of the hollow body dilates.

In another embodiment of the invention, the molecular alteration of the needle (2) nanomaterial is achieved by means of application of energy from an external device (not shown) which alters the needle (2) molecules through the modification of the electric or magnetic charge.

Now, in an embodiment of the invention, the needle (2) penetrates the vein of a donor through the use of a biotechnology sensor made of plastic or other nanomaterial, thus by having the ability to change the diameter, the needle (2) starts as a very small container at the moment of performing the penetration and grows in diameter when a command is sent (or after a predetermined time) to guarantee the painless blood donation, avoiding a discomfort in the patient or donor.

In another embodiment of the invention, the diameter change can be achieved with the command of nano-sensors which sense the gradient of the blood flow. Thus, a bioplastic material which is contained inside a nano-sensor senses the arrival of blood flow when contacts the vein. After a predetermined period of time, as short as a millisecond, the nano-sensor activates a structure change in the original diameter of the needle (2) and generates the enlargement of the polymer (latex) molecules being part thereof.

In this regard, once the nano-sensors of the needle (2) detect the interruption of blood flow, the material memory is activated returning to its original configuration. Thus, the needle (2) is removed from the vein of the patient with the same original size and it only grows when is inside the blood vessel.

Now, as previously stated, the change in the diameter of the needle (2) can be activated by means of an electric gradient, wherein a bioplastic material responds to a molecular configuration, mediated by an electric current. In this configuration, the needle (2) changes its diameter when the user activates an electric device near the puncture site, which alters the molecule distribution and the subsequent diameter.

Similarly, the diameter change can be achieved through induced magnetic fields, wherein the molecular configuration of a bioplastic changes when magnetic devices are activated during the blood donation or patient care.

According to the above, the polymers modified with nano and biotechnology can respond to temperature, electric, or magnetic changes or directly mediated by nano-sensors, such as those found in micro-robots, smart fabrics, and fibers and products related to space engineering.

Although the present invention has been disclosed in the form of preferred embodiments and variations thereon, it will be understood that numerous additional modifications and variations could be made thereto without departing from the scope of the invention.

For the sake of clarity, it is to be understood that the use of “a” or “an” throughout this application does not exclude a plurality, and “comprising” does not exclude other steps or elements. The mention of a “unit” or a “module” does not preclude the use of more than one unit or module.

1. A minimally invasive, biomedical device consisting of needle or catheter manufactured in a latex-derived nanomaterial which chemical composition is fundamentally based in fats, waxes and diverse gummy resins obtained from cytoplasm of lactic cells.

2. The device according to claim 1, further comprising:
a nanosensor located along the needle or catheter and generating an LEP-type optic stimulus along its path in order to activate the laboratory modified latex and which modifies its molecular structure;
a container for storing the liquid to be injected to removed;
a control system directly connected to the sensor; and
a power source which provides the necessary power to all the elements.

3. The device according to claim 1, wherein the nanosensor activates with a device which connected to the power source can provide the optic, electric or magnetic stimulus which generates the molecular modification of latex used in the manufacture of the needle or catheter.

4. The device according to claim 1, having a shutter which allows the activation and deactivation of the nanosensor at will of the health personnel.
